(19) United States
(12) Patent Application Publication
(10) Pub. No.: US 2025/0189540 A1
(43) Pub. Date: Jun. 12, 2025

(54) NOVEL BIOMARKER VISTA FOR DIAGNOSIS AND PROGNOSIS OF PATIENTS WITH IDIOPATHIC PULMONARY FIBROSIS

(71) Applicant: UNIVERSITY OF ULSAN FOUNDATION FOR INDUSTRY COOPERATION, Ulsan (KR)

(72) Inventor: Jin Woo SONG, Seoul (KR)

(73) Assignee: UNIVERSITY OF ULSAN FOUNDATION FOR INDUSTRY COOPERATION, Ulsan (KR)

(21) Appl. No.: 19/057,740

(22) Filed: Feb. 19, 2025

Related U.S. Application Data
(63) Continuation-in-part of application No. PCT/KR2023/012292, filed on Aug. 18, 2023.

(30) Foreign Application Priority Data
Aug. 19, 2022 (KR) 10-2022-0103691

(51) Int. Cl. G01N 33/68

(52) U.S. Cl. CPC G01N 33/6893; G01N 2333/70532; G01N 2800/12; G01N 2800/52

(57) ABSTRACT

A biomarker VISTA for the treatment, including diagnosis or prognosis, of patients having idiopathic pulmonary fibrosis. The biomarker VISTA allows a simple diagnosis or prognosis of idiopathic pulmonary fibrosis by analyzing the VISTA level in the blood isolated from a subject and exhibits an excellent effect on diagnosis and prognosis. The biomarker VISTA can be used as a non-invasive means for diagnosis or prognosis in the treatment of idiopathic pulmonary fibrosis.

### CROSS REFERENCE TO RELATED APPLICATIONS

This application is a continuation-in-part of PCT Application PCT/KR2023/012292, filed on Aug. 18, 2023, and claims priority to Korean Patent Application No. 10-2022-0103691, filed on Aug. 19, 2022, the entire contents of each of which are incorporated herein by reference.

### TECHNICAL FIELD

The present disclosure relates to a novel biomarker VISTA for diagnosis and prognosis of patients with idiopathic pulmonary fibrosis.

The present disclosure claims priority based on Korean Patent Application No. 10-2022-0103691, filed on Aug. 19, 2022, and all contents disclosed in the specifications and drawings of these applications are incorporated herein by reference.

### BACKGROUND ART

Idiopathic pulmonary fibrosis (IPF) is a fatal disease characterized by repeated inflammation in the lung interstitial tissue, leading to permanent scarring and tissue fibrosis. This results in structural changes in the lung tissue, ultimately causing impaired lung function and death. Approximately 5 million IPF patients occur worldwide, and in Korea, the incidence rate is 1.7 per 100,000 people. IPF accounts for more than 50% of interstitial lung diseases, making it the most commonly occurring condition among them.

IPF is a progressive disease that develops slowly over a period of one to two years, and as the disease progresses, it leads to the onset of respiratory difficulties. The average survival period is 60 months, but acute exacerbation occurs in 14% of patients annually. This disease shows low responsiveness to immunosuppressants and corticosteroids, and generally, treatment methods involve slowing the progression through pharmacological therapies such as antifibrotic agents.

Since the exact cause of IPF has not been identified, making treatment more challenging, it is crucial to detect the disease early through methods such as regular health check-ups. The diagnosis of IPF is made through typical chest CT imaging findings or surgical lung biopsy. However, in the case of chest imaging, there are often discrepancies in interpretation among readers, and in the case of surgical lung biopsy, it is often challenging to apply due to advanced age.

In order to solve the above-mentioned problem, research is being conducted on various diagnostic methods to develop methods that can be selected even in cases where conventional diagnostic methods, such as imaging or invasive tests, are difficult to apply. Blood tests are one of these methods. Blood tests have the advantage of enabling rapid acquisition of information regarding the occurrence and metastasis of diseases such as cancer by analyzing various biomarkers present in the blood from different parts of the body. VISTA (V-domain immunoglobulin suppressor of T-cell activation, PD-1H, Programmed Death One Homolog) is a molecule of the immunoglobulin superfamily and is primarily known to function as a co-inhibitor in the initiation of T cell responses to antigens. However, while there are studies suggesting its involvement in the diagnosis of a few cancers, its use for the diagnosis or prognosis prediction of idiopathic pulmonary fibrosis is not known.

### DISCLOSURE

### Technical Problem

To solve the above problem, the present disclosure develops the following methods of information provision, compositions, and kits.

One object of the present disclosure is to provide a method of information provision for diagnosing or predicting the prognosis of idiopathic pulmonary fibrosis, comprising detecting VISTA in blood separated from a subject.

Another object of the present disclosure is to provide a method of information provision for diagnosing or predicting the prognosis of idiopathic pulmonary fibrosis, comprising:
Detecting VISTA (V-domain Ig suppressor of T cell activation) in blood isolated from the subject; and
comparing the level of VISTA with the level of VISTA in blood isolated from a control group.

Another object of the present disclosure is to provide a composition for diagnosing or predicting the prognosis of idiopathic pulmonary fibrosis, comprising an agent for detecting VISTA.

Another object of the present disclosure is to provide a kit for diagnosing or predicting the prognosis of idiopathic pulmonary fibrosis, comprising a specification describing the method and the composition.

Another object of the present disclosure is to provide a method for detecting plasma biomarkers and treating idiopathic pulmonary fibrosis, comprising:
(a) detecting a level of VISTA(V-domain Ig suppressor of T cell activation) in plasma from a subject; and
(b) treating the subject for idiopathic pulmonary fibrosis when the level of VISTA is increased compared to a normal control group or a group of individuals who have been diagnosed with idiopathic pulmonary fibrosis at least once and have been cured;
wherein said treating comprises administering a therapeutically effective amount of a therapeutic agent.

However, the technical problems to be solved by the present disclosure are not limited to the problems mentioned above, and other unmentioned problems can be clearly understood by a person skilled in the art to which the present disclosure pertains from the description below.

### Technical Solution

To achieve the above objective, the present disclosure provides a method for providing information for the diagnosis or prognosis prediction of idiopathic pulmonary fibrosis, comprising.

In one embodiment of the present disclosure, wherein the method for providing information further comprises comparing the level of VISTA with the level of VISTA in blood separated from a normal control group, but is not limited thereto.

The present disclosure provides a composition for diagnosing or predicting the prognosis of idiopathic pulmonary fibrosis, comprising an agent for detecting VISTA.

In one embodiment of the present disclosure, the composition is capable of detecting VISTA in blood isolated from a subject, but is not limited thereto.

The present disclosure provides a diagnostic or prognostic kit for idiopathic pulmonary fibrosis, comprising a specification describing the method and the composition.

Additionally, the present disclosure provides a diagnostic or prognostic use for idiopathic pulmonary fibrosis of a composition comprising an agent for detecting VISTA as an active ingredient.

Additionally, the present disclosure provides a use for preparing a diagnostic or prognostic agent for idiopathic pulmonary fibrosis, of a composition comprising an agent for detecting VISTA as an active ingredient.

Additionally, the present disclosure provides a method for diagnosing idiopathic pulmonary fibrosis, comprising detecting VISTA in blood separated from a subject.

Additionally, the present disclosure provides a method for treating idiopathic pulmonary fibrosis, comprising detecting VISTA in the blood of a subject using an agent for detecting VISTA of the present disclosure; comparing the level of VISTA with that of a normal control group; and treating idiopathic pulmonary fibrosis.

Additionally, the present disclosure provides a method for predicting the prognosis of idiopathic pulmonary fibrosis in a subject, comprising detecting VISTA in the blood of a subject using an agent for detecting VISTA of the present disclosure; comparing the level of VISTA with that of a normal control group; and predicting the prognosis of idiopathic pulmonary fibrosis in the subject.

The present disclosure provides a method for providing information for the diagnosis or prognosis prediction of idiopathic pulmonary fibrosis, comprising:
Detecting VISTA (V-domain Ig suppressor of T cell activation) in the blood separated from the subject; and
comparing the level of VISTA with the level of VISTA in the blood separated from a control group.

In one embodiment of the present disclosure, the information providing method may further comprise diagnosing idiopathic pulmonary fibrosis or predicting a poor prognosis when the level of VISTA is increased compared to the level of VISTA in the control group, but is not limited thereto.

In one embodiment of the present disclosure, when the information providing method is for diagnosing idiopathic pulmonary fibrosis, the control group may include normal individuals or individuals who have been diagnosed with idiopathic pulmonary fibrosis at least once and have been cured, but is not limited thereto.

In one embodiment of the present disclosure, when the information providing method is for predicting the prognosis of idiopathic pulmonary fibrosis, the control group may include individuals who have been diagnosed with idiopathic pulmonary fibrosis at least once and have been cured, but is not limited thereto.

In one embodiment of the present disclosure, when the information providing method is for predicting the prognosis of idiopathic pulmonary fibrosis, the level of VISTA may be 320 μg/ml or higher, but is not limited thereto.

The present disclosure provides a composition for diagnosing or predicting the prognosis of idiopathic pulmonary fibrosis, comprising a composition for an agent for detecting VISTA.

In one embodiment of the present disclosure, the composition is capable of detecting the level of VISTA in blood separated from a subject, but is not limited thereto.

The present disclosure provides an idiopathic pulmonary fibrosis diagnostic or prognostic kit comprising a specification describing the method; and the composition.

Additionally, the present disclosure provides a method for treating idiopathic pulmonary fibrosis, comprising:
a) administering a biological agent to a subject;
b) determining the level of VISTA (V-domain Ig suppressor of T cell activation) in blood isolated from the subject;
c) comparing the level of VISTA with the level of VISTA in a control group; and
d) if the level of VISTA is decreased, re-administering the biological agent to the subject.

Additionally, the present disclosure provides a diagnostic device for the diagnosis or prognosis prediction of idiopathic pulmonary fibrosis, comprising a composition for detecting the level of VISTA in blood isolated from a subject as an active ingredient.

To achieve the above objective, the present disclosure provides a method for treating idiopathic pulmonary fibrosis, the method comprising:
determining a V-domain Ig suppressor of T cell activation (VISTA) level in plasma separated from a subject and comparing the detected VISTA level with a VISTA level determined in plasma separated from a control group; and
when the VISTA level in the subject's plasma is increased compared to the VISTA level in the control group, administering a therapeutically effective amount of a therapeutic agent to a subject diagnosed with idiopathic pulmonary fibrosis.

In one embodiment of the present disclosure, when the control group is a normal control group, wherein the administering is performed when
a mean VISTA level in the plasma of the subject is at least 1.76 times the VISTA level in the normal control group, and
a p-value for the mean VISTA level is less than 0.001, but is not limited thereto.

In one embodiment of the present disclosure, when the control group is a normal control group, wherein the administering is performed when a median VISTA level in the plasma of the subject is at least 1.6 times the VISTA level in the normal control group, but is not limited thereto.

In one embodiment of the present disclosure, when the control group is a normal control group, wherein the administering is performed when a maximum value of the VISTA level range in the plasma of the subject is at least 2.85 times the VISTA level in the control group, but is not limited thereto.

In one embodiment of the present disclosure, when the control group is a group who have been diagnosed with idiopathic pulmonary fibrosis at least once and cured, wherein the administering is performed when the level of VISTA in the plasma of the subject is greater than or equal to 320 μg/ml, but is not limited thereto.

To achieve the above objective, the present disclosure provides a method for detecting plasma biomarkers and treating idiopathic pulmonary fibrosis, comprising:
(a) detecting a level of VISTA(V-domain Ig suppressor of T cell activation) in plasma from a subject; and
(b) treating the subject for idiopathic pulmonary fibrosis when the level of VISTA is increased compared to a normal control group or a group of individuals who have been diagnosed with idiopathic pulmonary fibrosis at least once and have been cured;
wherein said treating comprises administering a therapeutically effective amount of a therapeutic agent.

In one embodiment of the present disclosure, wherein in step (b), when compared to the normal control group, the average level of VISTA in the subject's plasma is 1.76 times that of the normal control group, and the p-value for the average level of VISTA is less than 0.001, but is not limited thereto.

In one embodiment of the present disclosure, wherein in step (b), when compared to the normal control group, the median level of VISTA in the subject's plasma is 1.6 times that of the normal control group, but is not limited thereto.

In one embodiment of the present disclosure, wherein in step (b), when compared to the normal control group, the maximum value of the VISTA content range in the subject's plasma is 2.85 times that of the normal control group, but is not limited thereto.

In one embodiment of the present disclosure, wherein in step (b), when the control group consists of individuals who have been diagnosed with idiopathic pulmonary fibrosis at least once and cured, the level of VISTA in the plasma of the subject is greater than or equal to 320 μg/ml, but is not limited thereto.

### Advantageous Effects

The novel biomarker VISTA for diagnosis or prognosis of patients with idiopathic pulmonary fibrosis allows for the easy diagnosis and prognosis of idiopathic pulmonary fibrosis by analyzing the VISTA level in blood separated from the subject. Moreover, due to its excellent diagnostic and prognostic prediction effectiveness, it can be effectively used as a non-invasive method for diagnosing or predicting the prognosis of idiopathic pulmonary fibrosis.

### BRIEF DESCRIPTION OF THE DRAWINGS

FIG. 1 is a graph showing the ROC analysis results for the VISTA levels in survivors and deceased patients with idiopathic pulmonary fibrosis.

FIG. 2 is a Kaplan-Meier survival curve graph showing the survival rate according to the VISTA expression level.

### BEST MODE

The present disclosure relates to a method for diagnosing or predicting the prognosis of idiopathic pulmonary fibrosis (IPF) using VISTA (V-domain Ig suppressor of T cell activation) in blood isolated from a subject. According to one embodiment of the present disclosure, it was confirmed that the level of VISTA in blood is significantly higher in IPF patients compared to normal controls in the context of IPF diagnosis. Additionally, according to another embodiment of the present disclosure, a blood VISTA level of 320 μg/ml was identified as a cutoff value for IPF prognosis prediction, demonstrating excellent efficacy in both diagnosis and prognosis.

The present disclosure provides an information providing method for diagnosing or predicting the prognosis of idiopathic pulmonary fibrosis, comprising detecting VISTA (V-domain Ig suppressor of T cell activation) in blood isolated from a subject.

In one embodiment of the present disclosure, the information providing method may further include comparing the level of VISTA with the level of VISTA in blood isolated from a normal control group, but is not limited thereto.

Additionally, the method may further include determining idiopathic pulmonary fibrosis if the level of VISTA in the blood is higher than that of the normal control group, but is not limited thereto.

The present disclosure provides a method for providing information for the diagnosis or prognosis prediction of idiopathic pulmonary fibrosis, comprising:
detecting VISTA (V-domain Ig suppressor of T cell activation) in the blood separated from the subject; and
comparing the level of VISTA with the level of VISTA in the blood separated from a control group.

In one embodiment of the present disclosure, the information providing method may further include diagnosing idiopathic pulmonary fibrosis or predicting a poor prognosis when the level of VISTA is increased compared to the level of VISTA in the control group, but is not limited thereto.

In one embodiment of the present disclosure, when the information providing method is for diagnosing idiopathic pulmonary fibrosis, the control group may include normal individuals or individuals who have been diagnosed with idiopathic pulmonary fibrosis at least once and have been cured, but is not limited thereto.

In the present disclosure, normal subject may refer to a subject who has not been diagnosed with idiopathic pulmonary fibrosis, but is not limited thereto.

In one embodiment of the present disclosure, when the information providing method is for predicting the prognosis of idiopathic pulmonary fibrosis, the control group may include individuals who have been diagnosed with idiopathic pulmonary fibrosis at least once and have been cured, but is not limited thereto.

In the present disclosure, cure may refer to a condition in which the clinical symptoms of idiopathic pulmonary fibrosis no longer appear, allowing a diagnosis that the individual is no longer a patient with idiopathic pulmonary fibrosis in the relevant field. It may include cases where specific biomarkers indicative of idiopathic pulmonary fibrosis are absent or, if present, are at levels close to those of a normal subject, but is not limited thereto.

Additionally, in one embodiment of the present disclosure, when the information providing method is intended for predicting the prognosis of idiopathic pulmonary fibrosis, the level of VISTA in the blood may be 320 μg/ml or more, but is not limited thereto. Additionally, when the level of VISTA in the blood is 320 μg/ml or more, it may be determined that the prognosis of an idiopathic pulmonary fibrosis patient or subject is poor, but is not limited thereto.

In the present disclosure, “when the level of VISTA in the blood is 320 μg/ml or more” may refer to any value that can be detected as the level of VISTA in the subject's blood that is 320 μg/ml or higher, and is not specifically limited by the upper limit, nor does it necessarily make the content of the present disclosure unclear.

In the present disclosure, “poor prognosis” may refer to a lower survival rate (probability of survival) within a specific period (for example, a short-term period) compared to subjects with normal blood VISTA levels or those with blood VISTA levels above the normal range but below the cutoff value according to the present disclosure, the occurrence of two or more complications due to idiopathic pulmonary fibrosis, rapid deterioration of symptoms of idiopathic pulmonary fibrosis, or poor therapeutic response, but is not limited thereto. For example, in one embodiment of the present disclosure, a subject with a poor prognosis may reach death within 3 to 5 years compared to the normal control group, but is not limited thereto.

In the present disclosure, when the VISTA level in the blood is high, the subject's FVC (Forced Vital Capacity) or DLCO (Diffusing Capacity of the Lung for Carbon Monoxide) levels may be significantly low, but are not limited thereto. For example, in one embodiment of the present disclosure, when the VISTA level in the blood is high, pulmonary functional diseases, complications, metastasis related to FVC or DLCO, worsening of symptoms, or minimal therapeutic effects may occur, but are not limited thereto.

To achieve the above objective, the present disclosure provides a method for detecting plasma biomarkers and treating idiopathic pulmonary fibrosis, comprising:
(a) detecting a level of VISTA(V-domain Ig suppressor of T cell activation) in plasma from a subject; and
(b) treating the subject for idiopathic pulmonary fibrosis when the level of VISTA is increased compared to a normal control group or a group of individuals who have been diagnosed with idiopathic pulmonary fibrosis at least once and have been cured;
wherein said treating comprises administering a therapeutically effective amount of a therapeutic agent.

In one embodiment of the present disclosure, wherein in step (b), when compared to the normal control group, the average level of VISTA in the subject's plasma is 1.76 times that of the normal control group, and the p-value for the average level of VISTA is less than 0.001, but is not limited thereto.

In one embodiment of the present disclosure, wherein in step (b), when compared to the normal control group, the median level of VISTA in the subject's plasma is 1.6 times that of the normal control group, but is not limited thereto.

In one embodiment of the present disclosure, wherein in step (b), when compared to the normal control group, the maximum value of the VISTA content range in the subject's plasma is 2.85 times that of the normal control group, but is not limited thereto.

In one embodiment of the present disclosure, wherein in step (b), when the control group consists of individuals who have been diagnosed with idiopathic pulmonary fibrosis at least once and cured, the level of VISTA in the plasma of the subject is greater than or equal to 320 μg/ml, but is not limited thereto.

In an embodiment of the present invention, the mean VISTA concentration in the plasma of the IPF group may range from 350 to 450 μg/mL. For example, it may be in the range of 350 to 440 μg/mL, 350 to 430 μg/mL, 350 to 420 μg/mL, 350 to 410 μg/mL, 350 to 405 μg/mL, 350 to 403.24 pg/mL, 360 to 450 μg/mL, 360 to 440 μg/mL, 360 to 430 μg/mL, 360 to 420 μg/mL, 360 to 410 μg/mL, 360 to 405 μg/mL, 360 to 403.24 pg/mL, 370 to 450 μg/mL, 370 to 440 μg/mL, 370 to 430 μg/mL, 370 to 420 μg/mL, 370 to 410 μg/mL, 370 to 405 μg/mL, 370 to 403.24 pg/mL, 380 to 450 μg/mL, 380 to 440 μg/mL, 380 to 430 μg/mL, 380 to 420 μg/mL, 380 to 410 μg/mL, 380 to 405 μg/mL, 380 to 403.24 pg/mL, 390 to 450 μg/mL, 390 to 440 μg/mL, 390 to 430 μg/mL, 390 to 420 μg/mL, 390 to 410 μg/mL, 390 to 405 μg/mL, 390 to 403.24 pg/mL, 400 to 450 μg/mL, 400 to 440 μg/mL, 400 to 430 μg/mL, 400 to 420 μg/mL, 400 to 410 μg/mL, 400 to 405 μg/mL, 400 to 403.24 pg/mL, or 403.24 pg/mL, but is not limited to these ranges.

In another embodiment of the present invention, the median VISTA concentration in the plasma of the IPF group may range from 200 to 450 μg/mL. For example, it may be in the range of 200 to 445 μg/mL, 200 to 440 μg/mL, 200 to 435 μg/mL, 200 to 430 μg/mL, 200 to 425 μg/mL, 200 to 429.44 pg/mL, 205 to 450 μg/mL, 205 to 445 μg/mL, 205 to 440 μg/mL, 205 to 435 μg/mL, 205 to 430 μg/mL, 205 to 425 μg/mL, 205 to 429.44 pg/mL, 210 to 450 μg/mL, 210 to 445 μg/mL, 210 to 440 μg/mL, 210 to 435 μg/mL, 210 to 430 μg/mL, 210 to 425 μg/mL, 210 to 429.44 pg/mL, 215 to 450 μg/mL, 215 to 445 μg/mL, 215 to 440 μg/mL, 215 to 435 μg/mL, 215 to 430 μg/mL, 215 to 425 μg/mL, 215 to 429.44 pg/mL, 220 to 450 μg/mL, 220 to 445 μg/mL, 220 to 440 pg/mL, 220 to 435 μg/mL, 220 to 430 μg/mL, 220 to 425 μg/mL, 220 to 429.44 pg/mL, 425 to 450 μg/mL, 425 to 440 μg/mL, 425 to 430 μg/mL, 425 to 420 μg/mL, 425 to 410 μg/mL, 425 to 405 μg/mL, 425 to 429.44 pg/mL, but is not limited to these ranges.

Furthermore, the median concentration of VISTA may typically range from 260 to 360 μg/mL, for example, 260 to 350 μg/mL, 260 to 340 μg/mL, 260 to 330 μg/mL, 260 to 320 μg/mL, 260 to 312.52 pg/mL, 270 to 360 μg/mL, 270 to 350 μg/mL, 270 to 340 μg/mL, 270 to 330 μg/mL, 270 to 320 μg/mL, 270 to 312.52 pg/mL, 280 to 360 μg/mL, 280 to 350 μg/mL, 280 to 340 μg/mL, 280 to 330 μg/mL, 280 to 320 μg/mL, 280 to 312.52 pg/mL, 290 to 360 μg/mL, 290 to 350 μg/mL, 290 to 340 μg/mL, 290 to 330 μg/mL, 290 to 320 μg/mL, 290 to 312.52 pg/mL, 300 to 360 μg/mL, 300 to 350 μg/mL, 300 to 340 μg/mL, 300 to 330 μg/mL, 300 to 320 μg/mL, 300 to 312.52 pg/mL, 310 to 360 μg/mL, 310 to 350 μg/mL, 310 to 340 μg/mL, 310 to 330 μg/mL, 310 to 320 μg/mL, 310 to 312.52 pg/mL, or 312.52 pg/mL, but is not limited to these ranges.

In a further embodiment of the present invention, the range of VISTA concentrations in the plasma of the IPF group may be from 50 to 3200 μg/mL. For example, it may range from 50 to 3180 μg/mL, 50 to 3170 μg/mL, 50 to 3160 μg/mL, 50 to 3150 μg/mL, 50 to 3139.44 pg/mL, 60 to 3200 μg/mL, 60 to 3180 μg/mL, 60 to 3170 μg/mL, 60 to 3160 μg/mL, 60 to 3150 μg/mL, 60 to 3139.44 pg/mL, 70 to 3200 μg/mL, 70 to 3180 μg/mL, 70 to 3170 μg/mL, 70 to 3160 μg/mL, 70 to 3150 μg/mL, 70 to 3139.44 pg/mL, 80 to 3200 μg/mL, 80 to 3180 μg/mL, 80 to 3170 μg/mL, 80 to 3160 μg/mL, 80 to 3150 μg/mL, 80 to 3139.44 pg/mL, 90 to 3200 μg/mL, 90 to 3180 μg/mL, 90 to 3170 μg/mL, 90 to 3160 μg/mL, 90 to 3150 μg/mL, 90 to 3139.44 pg/mL, 100 to 3200 μg/mL, 100 to 3180 μg/mL, 100 to 3170 μg/mL, 100 to 3160 μg/mL, 100 to 3150 μg/mL, 100 to 3139.44 pg/mL, 105 to 3200 μg/mL, 105 to 3180 μg/mL, 105 to 3170 μg/mL, 105 to 3160 μg/mL, 105 to 3150 μg/mL, 105 to 3139.44 pg/mL, 109.2 to 3200 μg/mL, 109.2 to 3180 μg/mL, 109.2 to 3170 μg/mL, 109.2 to 3160 μg/mL, 109.2 to 3150 μg/mL, or 109.2 to 3139.44 pg/mL, but is not limited to these ranges.

In the present disclosure, “VISTA (V-domain Ig suppressor of T cell activation)” refers to a novel immune checkpoint(ICs) expressed in bone marrow cells, lymphocytes, and tumor cells, also known as PD-1H(Programmed-1h), which substantially regulates both innate and adaptive anti-tumor immune responses.

In the present disclosure, “blood” refers to the blood separated from a subject for analysis, and may, for example, be collected from the veins near the elbow or from the fingertip. However, it is not limited thereto and can be separated using generally accepted collection methods depending on the amount needed for analysis.

When collecting blood samples for analysis, whole blood, serum, or plasma may be collected. However, it is not limited thereto, and any form that allows the application of the information providing method for diagnosis or prognosis prediction according to the present disclosure is acceptable.

The blood collection device refers to a device commonly used for collecting blood from a subject, and depending on the type of collection device, additional devices may be included to prevent the collection of unnecessary contaminants or foreign substances along with the blood, but is not limited thereto.

The collected blood may be preprocessed to facilitate biomarker detection, but is not limited thereto. For example, homogenization, filtration, distillation, extraction, concentration, and cooling processes may be applied for temperature or humidity control, removal of unwanted impurities, etc. Additional processes or reagents may be applied to deactivate interfering components, and reagents such as anticoagulants may be added, but is not limited thereto.

The collected blood may be analyzed immediately after being separated from the subject, or it may be stored for a certain period before analysis. In this case, necessary substances may be added for storage, but is not limited thereto.

Additionally, the present disclosure provides a composition for diagnosing or predicting the prognosis of idiopathic pulmonary fibrosis, comprising an agent for detecting VISTA.

In the present disclosure, the VISTA is a biomarker, and the agent for detecting VISTA may be a protein, polynucleotide, nucleic acid, compound, antibody, aptamer, or the like, but is not limited thereto. Any form of reagent that can generally be used to detect the VISTA may be applied.

In the present disclosure, the term “biomarker” refers to a marker that can distinguish between normal and pathological conditions, predict treatment responses, and be objectively measured. The term “idiopathic pulmonary fibrosis biomarker” refers to cells, proteins, DNA, RNA, metabolites, and other substances that can be used to distinguish and diagnose patients with idiopathic pulmonary fibrosis from biological samples of the subject. The present disclosure provides a biomarker for distinguishing idiopathic pulmonary fibrosis patients from a normal control group or an interstitial lung disease group.

In the present disclosure, the term “protein” is used interchangeably with “polypeptide” or “peptide,” and refers to a polymer of amino acid residues, as typically found in naturally occurring proteins.

In the present disclosure, the term “polynucleotide” or “nucleic acid” refers to deoxyribonucleic acid (DNA) or ribonucleic acid (RNA) in the form of single-stranded or double-stranded molecules. Unless otherwise limited, the term “nucleic acid” also includes known analogs of naturally occurring nucleotides that hybridize with nucleic acids in a manner similar to naturally occurring nucleotides.

In the present disclosure, the term “antibody” refers to a specific protein molecule directed against an antigenic site. For the purposes of the present disclosure, the term “antibody” refers to an antibody that specifically binds to a marker protein, and includes polyclonal antibodies, monoclonal antibodies, and recombinant antibodies. Additionally, any portion of an antibody that retains antigen-antibody binding ability is included as an antibody of the present disclosure, and all types of immunoglobulin antibodies that specifically bind to the exhaled biomarkers presented in the present disclosure are included. For example, the complete form of an antibody having two full-length light chains and two full-length heavy chains, as well as functional fragments of the antibody molecule, including Fab, F(ab′), F(ab′)2, and Fv, which possess antigen-binding functionality, are included. Furthermore, the antibodies of the present disclosure include special antibodies such as humanized antibodies, chimeric antibodies, and recombinant antibodies, as long as they can specifically bind to the proteins of the present disclosure.

In the present disclosure, the term “aptamer” refers to a single-stranded nucleic acid (DNA, RNA, or modified nucleic acid) that has a stable tertiary structure and can specifically bind to an analyte to be detected in a sample, thereby enabling the specific detection of the presence of a target protein in the sample. The preparation of the aptamer can be performed according to conventional methods for aptamer production, which involves determining and synthesizing the sequence of oligonucleotides with selective and high binding affinity for the target protein to be detected. Subsequently, the 5′ or 3′ end of the oligonucleotide can be modified with —SH, —COOH, —OH, or NH₂ groups to enable binding to functional groups on the aptamer chip, but is not limited thereto.

The an agent for detecting VISTA may include salts, compounds, substances that serve additional functional roles, and the like, which can all be used generally in the preparation. These substances may be present separately for addition when using the kit, but are not limited thereto.

In one embodiment of the present disclosure, the agent for detecting VISTA may detect the level of VISTA in the blood separated from the subject.

Additionally, the present disclosure provides a kit for diagnosing or predicting the prognosis of idiopathic pulmonary fibrosis, comprising the specification describing the method and the composition.

In the present disclosure, “detection” may include quantifying the concentration of the subject, and it also encompasses a qualitative meaning (qualitative analysis) to confirm the presence or absence of a specific substance. Therefore, it refers to both measuring and confirming the presence (expression) of the intended substance, as well as measuring and confirming changes in the presence level (expression level) of the intended substance.

In the present disclosure, “level” is preferably determined by “measurement.” The qualitative analysis may refer to measuring and confirming the presence or absence of the intended substance, while the quantitative analysis may refer to measuring and confirming changes in the level (expression level) or quantity of the intended substance. In the present disclosure, level analysis can be performed without limitation using both qualitative and quantitative methods, and may involve performing quantitative measurement.

In the present disclosure, the term “diagnosis” refers to determining the susceptibility of a subject to a specific disease or condition, determining whether the subject currently has a specific disease or condition, assessing the prognosis of a subject with a specific disease or condition, or including therametrics (e.g., monitoring the status of an object to provide information about therapeutic efficacy).

In the present invention, “prognostic prediction” or “prognosis” refers to the prediction of the disease progression in a patient population with the disease of the present invention. It can refer to predicting the probabilities of disease progression, worsening, relapse, or stability, etc., through the increase or decrease in the levels of the biomarkers of the present invention.

In the present disclosure, the term “kit” refers to a tool that enables distinguishing idiopathic pulmonary fibrosis patients from a normal control group. The kit of the present disclosure may include an agent for detecting VISTA, as well as other components, compositions, solutions, and devices typically required for the detection method. There are no limitations on the order in which these substances are applied, and the application of each substance may occur simultaneously or sequentially.

In the present disclosure, the kit may further include a container or the like, but is not limited thereto.

The container may serve the role of packaging the substances, as well as the role of storing and securing them. The material of the container may be, for example, plastic, glass bottles, etc., but is not limited thereto.

Additionally, the present disclosure provides a method for treating idiopathic pulmonary fibrosis, comprising detecting VISTA in the blood of a subject using an agent for detecting VISTA of the present disclosure; comparing the level of VISTA with that of a normal control group; and treating idiopathic pulmonary fibrosis.

In the present disclosure, the “method for treating idiopathic pulmonary fibrosis” may be applied simultaneously or sequentially with general treatment methods for treating idiopathic pulmonary fibrosis, but is not limited thereto.

In the present disclosure, the “method for treating idiopathic pulmonary fibrosis” may involve the co-prescription of a prophylactic or therapeutic composition for preventing or treating idiopathic pulmonary fibrosis.

The pharmaceutical composition for prevention or treatment of the present disclosure may further include an appropriate carrier, excipient, and diluent commonly used in the preparation of pharmaceutical compositions.

The excipient may be one or more selected from the group consisting of a diluent, binder, disintegrant, lubricant, adsorbent, humectant, film-coating material, and controlled-release additive.

The pharmaceutical composition of the present disclosure can be formulated and used in various forms, including powders, granules, sustained-release granules, enteric-coated granules, liquids, eye drops, elixirs, emulsions, suspensions, tinctures, troches, aromatic waters, limonades, tablets, sustained-release tablets, enteric-coated tablets, sublingual tablets, hard capsules, soft capsules, sustained-release capsules, enteric-coated capsules, lozenges, tinctures, soft extracts, dry extracts, liquid extracts, injections, capsules, perfusates, suppositories, lotions, pastes, sprays, inhalants, patches, sterilized injectable solutions, or aerosols, and the external preparations may include forms such as creams, gels, patches, sprays, ointments, lotions, liniments, pastes, or cataplasms.

The carriers, excipients, and diluents that can be included in the pharmaceutical composition of the present disclosure include lactose, dextrose, sucrose, oligosaccharides, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methylhydroxybenzoate, propylhydroxybenzoate, talc, magnesium stearate, and mineral oil.

When formulating the preparation, diluents or excipients such as commonly used fillers, extenders, binders, wetting agents, disintegrants, surfactants, and similar substances are used.

The excipients that may be used in the tablets, powders, granules, capsules, pills, and troches of the present disclosure include excipients such as corn starch, potato starch, wheat starch, lactose, sucrose, glucose, fructose, D-mannitol, precipitated calcium carbonate, synthetic aluminum silicate, calcium dihydrogen phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropylmethylcellulose (HPMC), HPMC 1828, HPMC 2906, HPMC 2910, propylene glycol, casein, calcium lactate, Primojel, etc. as diluents; gelatin, gum arabic, ethanol, agar powder, cellulose acetate phthalate, carboxymethylcellulose, calcium carboxymethylcellulose, glucose, purified water, sodium caseinate, glycerin, stearic acid, sodium carboxymethylcellulose, methylcellulose sodium, methylcellulose, microcrystalline cellulose, dextrin, hydroxycellulose, hydroxypropyl starch, hydroxy-methylcellulose, purified shellac, starch powder, hydroxypropyl cellulose, hydroxypropylmethylcellulose, polyvinyl alcohol, polyvinylpyrrolidone, etc. as binders; hydroxypropylmethylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, anhydrous silica, 1-hydroxypropyl cellulose, dextran, ion exchange resin, polyvinyl acetate, formaldehyde-treated casein and gelatin, alginate, amylose, guar gum, sodium bicarbonate, polyvinylpyrrolidone, calcium phosphate, gel-forming starch, gum arabic, amylopectin, pectin, sodium polyphosphate, ethylcellulose, sucrose, magnesium aluminum silicate, D-sorbitol solution, and anhydrous silica as disintegrants; calcium stearate, magnesium stearate, stearic acid, hydrogenated vegetable oil, talc, kaolin, baselin, sodium stearate, cocoa butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, macrogol, synthetic aluminum silicate, anhydrous silica, long-chain fatty acids, high alcohols, silicone oil, paraffin oil, polyethylene glycol fatty acid esters, starch, sodium chloride, sodium acetate, sodium oleate, dl-leucine, and anhydrous silica as lubricants.

The excipients that may be used in the liquid preparations of the present disclosure include water, dilute hydrochloric acid, dilute sulfuric acid, sodium citrate, monostearate sucrose, polyoxyethylene sorbitan fatty acid esters (Tween esters), polyoxyethylene monoalkyl ethers, lanolin ethers, lanolin esters, acetic acid, hydrochloric acid, ammonia solution, ammonium bicarbonate, potassium hydroxide, sodium hydroxide, prolamine, polyvinylpyrrolidone, ethylcellulose, sodium carboxymethylcellulose, and similar substances.

The syrup of the present disclosure may be used in the solution of sucrose, other sugars, or sweeteners, and similar substances, and may be used, as needed, flavoring agents, coloring agents, preservatives, stabilizers, suspending agents, emulsifiers, thickeners, and similar substances.

The emulsion of the present disclosure may be used in purified water, and may be used, as needed, emulsifiers, preservatives, stabilizers, flavoring agents, and similar substances.

The suspension of the present disclosure may be used in suspending agents such as acacia, tragacanth, methylcellulose, carboxymethylcellulose, carboxymethylcellulose sodium, microcrystalline cellulose, sodium alginate, hydroxypropylmethylcellulose (HPMC), HPMC 1828, HPMC 2906, HPMC 2910, and similar substances, and may be used, as needed, surfactants, preservatives, stabilizers, colorants, flavoring agents, and similar substances.

The injectable formulations of the present disclosure may include solvents such as purified water for injection, 0.9% sodium chloride injection, Ringer's injection, dextrose injection, dextrose+sodium chloride injection, PEG (polyethylene glycol), lactated Ringer's injection, ethanol, propylene glycol, non-volatile oils such as sesame oil, cottonseed oil, rapeseed oil, soybean oil, corn oil, ethyl oleate, isopropyl myristate, and benzyl benzoate; solubilizing agents such as sodium benzoate, sodium salicylate, sodium acetate, urea, urethane, monoethyl acetamide, butazolidine, propylene glycol, Tween, nicotinic acid amide, hexamine, dimethylacetamide; buffering agents such as weak acids and their salts (acetic acid and sodium acetate), weak bases and their salts (ammonia and ammonium acetate), organic compounds, proteins, albumin, peptones, gums; stabilizing agents such as sodium chloride, stabilizers such as sodium bisulfite (NaHSO3), carbon dioxide gas, sodium metabisulfite (Na2S2O5), sodium sulfite (Na2SO3), nitrogen gas (N2), ethylenediaminetetraacetic acid; antioxidants such as sodium bisulfite 0.1%, sodium formaldéhyde sulfoxylate, thiourea, ethylenediaminetetraacetic acid disodium, acetone sodium bisulfite; analgesics such as benzyl alcohol, chlorobutanol, procaine hydrochloride, glucose, calcium gluconate; and suspending agents such as CMC sodium, sodium alginate, Tween 80, and aluminum monostearate.

The suppository of the present disclosure may be used in bases such as cocoa butter, lanolin, Witepsol, polyethylene glycol, glycerogelatin, methylcellulose, carboxymethylcellulose, a mixture of stearic acid and oleic acid, Subanal, cottonseed oil, safflower oil, palm oil, cocoa butter+cholesterol, lecithin, lanette wax, monostearin glycerol, Tween or Span, Imhausen, Monolen (monostearic acid propylene glycol), glycerin, Adeps solidus, Buytyrum Tego-G, Cebes Pharma 16, Hexaride Base 95, Cotomar, Hydrokote SP, S-70-XXA, S-70-XX75 (S-70-XX95), Hydrokote 25, Hydrokote 711, Idropostal, Massa estrarium (A, AS, B, C, D, E, I, T), Massa-MF, Masupol, Masupol-15, Neosufostal-N, Paramount-B, Suposhiro (OSI, OSIX, A, B, C, D, H, L), Suppository Base IV Type (AB, B, A, BC, BBG, E, BGF, C, D, 299), Supostal (N, Es), Weco (W, R, S, M, Fs), and Tegester Triglyceride Base (TG-95, MA, 57), and similar substances.

The solid dosage forms for oral administration of the present disclosure may include tablets, powders, granules, and capsules, and such solid dosage forms are prepared by mixing the above-mentioned extract with at least one excipient, such as starch, calcium carbonate, sucrose, lactose, gelatin, and similar substances. Additionally, lubricants such as magnesium stearate and talc may be used, aside from simple excipients.

The liquid formulations for oral administration of the present disclosure may include suspensions, solutions, emulsions, syrups, and similar substances, and may also include various excipients, such as wetting agents, sweeteners, flavoring agents, preservatives, and similar substances, in addition to commonly used simple diluents such as water and liquid paraffin. The formulations for non-oral administration include sterilized aqueous solutions, non-aqueous solvents, suspensions, emulsions, lyophilized formulations, and suppositories. The non-aqueous solvents and suspending agents of the present disclosure may be used in propylene glycol, polyethylene glycol, vegetable oils such as olive oil, and injectable esters such as ethyl oleate, and similar substances.

The pharmaceutical composition of the present disclosure is administered in an amount effective for pharmacological activity. In the present disclosure, the term “pharmaceutically effective amount” refers to an amount sufficient to treat the disease with a reasonable benefit/risk ratio applicable to medical treatment, and the effective dosage level can be determined based on factors such as the type and severity of the patient's condition, the activity of the drug, the patient's sensitivity to the drug, the administration time, the route of administration, the elimination rate, the treatment duration, other drugs used concomitantly, and other factors well-known in the medical field.

The pharmaceutically acceptable composition of the present disclosure may be administered as an individual therapeutic agent or in combination with other therapeutic agents, and may be administered sequentially or simultaneously with conventional therapeutic agents, either as a single or multiple doses. It is important to administer an amount that provides the maximum effect with the minimum amount without side effects, considering all the aforementioned factors, and this can be easily determined by a person skilled in the art to which the present disclosure pertains.

The pharmaceutical composition of the present disclosure may be administered to an individual via various routes. All methods of administration are foreseeable, including, for example, oral administration, subcutaneous injection, intraperitoneal administration, intravenous injection, intramuscular injection, intrathecal injection (epidural space), sublingual administration, buccal administration, rectal insertion, vaginal insertion, ocular administration, otic administration, nasal administration, inhalation, spraying through the mouth or nose, transdermal administration, and other forms of administration.

The pharmaceutical composition of the present disclosure is determined based on various relevant factors, including the disease to be treated, the administration route, the patient's age, gender, weight, severity of the disease, and the type of active ingredient.

In the present disclosure, the term “subject” refers to an individual in need of treatment for a disease, and more specifically includes mammals such as humans, non-human primates, mice, rats, dogs, cats, horses, and cattle.

In the present disclosure, the term “administration” refers to providing a predetermined composition of the present disclosure to a subject by any appropriate method.

In the present disclosure, the term “prevention” refers to any act of inhibiting or delaying the onset of a target disease, and the term “treatment” refers to any act by which the target disease and its associated metabolic abnormalities are improved or favorably altered by the administration of the pharmaceutical composition of the present disclosure. The term “improvement” refers to any act of reducing the severity of symptoms or other parameters related to the target disease through the administration of the composition of the present disclosure.

The present disclosure provides a method for screening a therapeutic agent for idiopathic pulmonary fibrosis, comprising:
(a) detecting the level of VISTA isolated plasma from an idiopathic pulmonary fibrosis model treated with a candidate substance; and
(b) selecting the candidate substance as a therapeutic agent for idiopathic pulmonary fibrosis if the level of VISTA is decreased in the plasma from the subject.

In the present invention, the term “screening” refers to the process of selecting a substance with a specific property from a group of candidate substances through a particular manipulation or evaluation method.

Specifically, for the purposes of the present disclosure, the screening method involves a series of steps to assess the efficacy of candidate drug substances using the method described above, in order to identify the most effective therapeutic agent for idiopathic pulmonary fibrosis, particularly for individuals with poor prognosis predicted to have solid tumors, although it is not limited to this.

The detecting the therapeutic response and effect can be repeated several times depending on the candidate substance, and additional substances or steps may be included for further confirmation of therapeutic response and effect. These steps are common in screening methods and can include additional procedures as conventionally used in the art, but this is not restrictive.

In the present invention, the term “candidate substance” refers to an unknown substance used in the screening process to measure the expression changes of the biomarker in the disease model of the invention. It can be selected from the group consisting of nucleotides, DNA, RNA, amino acids, aptamers, proteins, stem cells, stem cell culture media, compounds, microbial culture media or extracts, natural products, and natural extracts, but is not limited to these.

In the present invention, the term “treatment” refers to any action that improves or beneficially alters the condition of the targeted disease and its related metabolic abnormalities. This includes the use of chemotherapy, surgical procedures, biological therapy, and other methods.

In this context, if the levels of the biomarkers of the present invention in the substance being tested, compared to a control group (such as a normal control group), increase or decrease depending on the type of biomarker, it can be concluded that the targeted disease has been treated (or improved). As described above, “increased levels” refer to the previously mentioned criteria.

For the treatment of the targeted disease in the present invention, conventional therapeutic methods or conventional therapeutic drugs can be used, or the candidate substances disclosed in the present invention may be administered, but this is not restrictive.

In the present specification, the term “increased levels” refers to either the detection of a substance that was previously undetectable or an increase in the amount detected relative to normal levels.

For example, an “increase” in levels means that the level in the experimental group is at least 1%, 2%, 3%, 4%, 5%, 10%, or more, relative to the level in the control group. Specifically, it can mean an increase of 5%, 10%, 20%, 30%, 40%, 50%, 60%, 70%, 80%, 90%, or more, and/or an increase of 0.5-fold, 1.1-fold, 1.2-fold, 1.4-fold, 1.6-fold, 1.8-fold, or more.

More specifically, the increase may range from 1 to 1.5 times, 1.5 to 2 times, 2 to 2.5 times, 2.5 to 3 times, 3 to 3.5 times, 3.5 to 4 times, 4 to 4.5 times, 4.5 to 5 times, 5 to 5.5 times, 5.5 to 6 times, 6 to 6.5 times, 6.5 to 7 times, 7 to 7.5 times, 7.5 to 8 times, 8 to 8.5 times, 8.5 to 9 times, 9 to 9.5 times, 9.5 to 10 times, or 10 times or more compared to the control group. However, this is not restrictive.

The meaning of the opposing term can be understood by a person skilled in the art as being the opposite of the above definition, consistent with the opposite meaning.

The terms used in the present disclosure have been selected based on commonly used general terms currently prevalent, considering the functions within the context of the present disclosure. However, these terms may vary depending on the intent of those skilled in the art, case law, or the emergence of new technologies. Additionally, in certain cases, terms may be arbitrarily selected by the applicant, and in such cases, their meaning will be described in detail in the specification of the relevant invention. Therefore, the terms used in the present disclosure should be defined based not merely on the names of the terms themselves, but on their meanings and the content of the present disclosure as a whole.

In the specification of the present disclosure, when a certain part is said to “include” a particular component, it means that, unless explicitly stated otherwise, it is not excluding other components, but may include additional components. In the entire specification of the present disclosure, terms such as “about” and “substantially” are used to mean within the manufacturing and material tolerances inherent in the mentioned meaning, or close to the specified values, and are used to prevent unscrupulous infringers from unfairly exploiting the disclosed content where exact or absolute values are mentioned, in order to aid in the understanding of the present disclosure.

In the entire specification of the present disclosure, the term “combination thereof” included in the Markush-type expression refers to one or more mixtures or combinations selected from the group of components described in the Markush-type expression, meaning that it includes one or more selected from the group consisting of the components described above.

### Modes of the Invention

Hereinafter, preferred examples are presented to aid in understanding the present disclosure. However, the following examples are provided merely for easier understanding of the present disclosure, and the scope of the present disclosure is not limited by these examples.

### EXAMPLE

### Example 1. Verification of the Diagnostic Use of VISTA for Idiopathic Pulmonary Fibrosis

To verify the use of VISTA for diagnosing idiopathic pulmonary fibrosis, the VISTA content in the blood of an idiopathic pulmonary fibrosis patient group (IPF) and a normal control group (Control) was measured using the ELISA method. Specifically, 100 individuals were recruited for the idiopathic pulmonary fibrosis patient group and 60 individuals for the normal control group (Table 1). The data were presented as mean±standard deviation or number (%), where BMI refers to body mass index, FVC (forced vital capacity) refers to forced vital capacity, and DLCO (diffusing capacity of the lung for carbon monoxide (CO)) refers to lung diffusion capacity.

TABLE 1

| Classification | IPF | Control | p value |
|---|---|---|---|
| Number of patients | 100 | 60 | |
| Age (years) | 67.8 ± 7.8 | 63.7 ± 3.6 | <0.001 |
| Gender ratio | 79/11 | 54/6 | 0.072 |
| (Male:Female) | | | |
| BM(kg/m²) | 22.9 ± 2.2 | — | — |
| FVC(%, predicted) | 64.3 ± 16.3 | — | — |
| DLCO(%, predicted) | 48.6 ± 19.3 | — | — |

Plasma samples were collected from a total of 160 recruited individuals. Specifically, plasma samples from IPF patients were collected at Asan Medical Center in South Korea with prior consent under the approval of the Institutional Review Board (IRB) of Asan Medical Center (IRB No. 2016-1131). All IPF patients met the diagnostic criteria for IPF outlined in the 2018 statement by the American Thoracic Society (ATS), European Respiratory Society (ERS), Japanese Respiratory Society, and Latin American Thoracic Association. The plasma samples for the control group were provided by the Ajou University Hospital Biobank (Approval No.: AJHB-2021-31) and the Seoul National University Bundang Hospital Human Biobank (Approval No.: DT-2021-009-01). The control group was identified through medical history and health check-ups, and written consent was obtained for the use of their samples in future studies. Plasma samples collected in BD Vacutainer EDTA tubes (BD #367844) were centrifuged at 3000 rpm and 4° C. for 10 minutes (Eppendorf #5810 R) and stored at −70° C. until analysis.

To measure the VISTA levels in the collected plasma samples, the VISTA DuoSet ELISA Kit (R&D Systems, #DY7126) and the DuoSet Ancillary Reagent Kit (R&D Systems, #DY008) were used according to the manufacturer's instructions. Specifically, 100 mL of plasma sample per well was applied to ELISA in duplicate for each well. The absorbance (A450-A540) readings were performed using a SpectraMax190 (Molecular devices) ELISA reader, and the VISTA levels in the samples were calculated by generating a 4-parameter logistic curve-fit.

TABLE 2

| Classification | | IPF | Control | p value |
|---|---|---|---|---|
| VISTA | Mean ± SD | 403.24 ± 413.84 | 229.54 ± 145.87 | <0.001 |
| (pg/ [symbol] ) | Median | 312.52 | 195.23 | — |
| | (IQR) | (225.42-429.44) | (169.12-224.16) | |
| | Range | 109.20-3139.44 | 119.28-1121.42 | — |

* Cohort total: IPF(N = 100) and Control(N = 60)

As a result, the mean value of VISTA in the idiopathic pulmonary fibrosis patient group was 1.76 times higher compared to the normal control group, which was found to be statistically significant. Additionally, the median value of VISTA in the idiopathic pulmonary fibrosis patient group was approximately 1.6 times higher. Furthermore, the minimum level range of VISTA in the idiopathic pulmonary fibrosis patient group showed little difference from the normal control group, but the maximum value was found to be approximately three times higher (IPF=3139.44, Control=1121.42) (Table 2).

According to these results, the VISTA levels in the blood samples collected from the idiopathic pulmonary fibrosis patient group were significantly higher compared to the normal control group, and this was statistically significant (p value=<0.001). Thus, it was confirmed that VISTA can be used as a blood biomarker for idiopathic pulmonary fibrosis.

### Example 2. Confirmation of the Prognostic Use of VISTA for Idiopathic Pulmonary Fibrosis Through ROC Analysis

In order to confirm the prognostic use of VISTA for of idiopathic pulmonary fibrosis and determine the optimal cut-off value, time-dependent ROC (receiver operating characteristic) analysis was performed. Specifically, the area under the ROC curve (AUC) was used as a measure to evaluate how well it distinguishes between IPF deceased and surviving patients, and the cut-off for future classification was selected. In this case, the cut-off is a single score that minimizes sensitivity while maximizing specificity, and it was analyzed using SPSS Statistics (version 24.0; IBM Corp., Armonk, NY, USA) and R Statistics software version 4.0.2 (The R Foundation, Vienna, Austria).

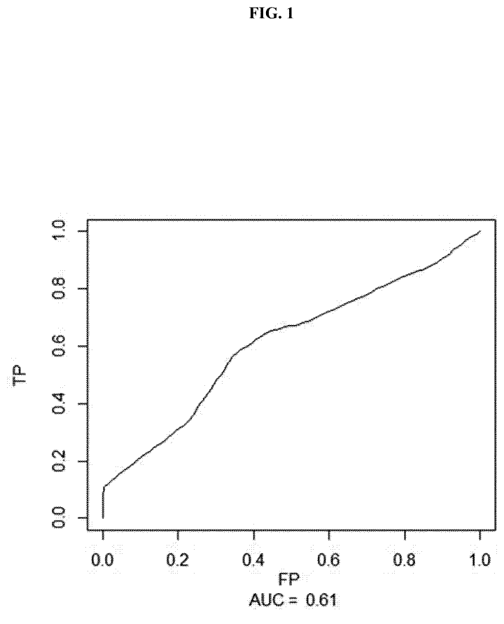

According to the above method, survival of idiopathic pulmonary fibrosis patients was tracked for 19 months (median, interquartile range 8-37 months). As a result, VISTA levels were higher in the deceased (n=53) than in the survivors (n=47) (FIG. 1). At this time, the AUC was 0.61 and the p-value was 0.063, indicating that VISTA levels in plasma can be effectively used to classify idiopathic pulmonary fibrosis patients. The optimal cutoff value at this point was found to be 320 pg/ml.

### Example 3. Confirmation of the Prognistic Use of VISTA for Idiopathic Pulmonary Fibrosis Through Kaplan-Meier Survival Curve Analysis

To confirm the use of VISTA for prognostic prediction of idiopathic pulmonary fibrosis, Kaplan-Meier survival curve analysis was performed using R Statistics software version 4.0.2 (The R Foundation, Vienna, Austria).

TABLE 3

| | 0 | 1 year | 2 year | 3 year | 5 year |
|---|---|---|---|---|---|
| VISTA Level | (persons) | (persons) | (persons) | (persons) | (persons) |
| <320 pg/mL(0) | 55 | 46 | 36 | 35 | 34 |
| ≥320 pg/mL(1) | 45 | 28 | 21 | 20 | 19 |
| p value | | 0.029 | 0.087 | 0.084 | 0.097 |

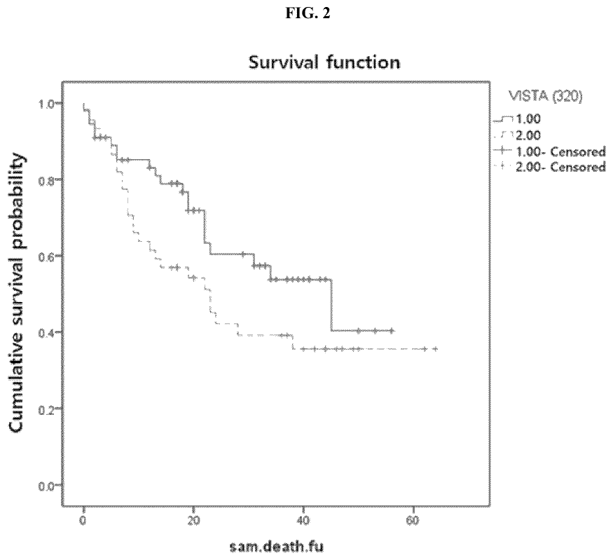

In the Kaplan-Meier survival curve analysis, the number of IPF patients after 5 years was classified according to the optimal cutoff value of plasma VISTA levels (FIG. 2). The group with high VISTA expression (≥320 pg/ml) consisted of 19 out of 45 total patients (42.2%), while the group with low VISTA expression (<320 pg/ml) consisted of 34 out of 55 total patients (Table 3). The group with high VISTA expression was found to have a lower survival probability compared to the group with low VISTA expression (3-year survival rate: adjusted HR, 1.908; 95% confidence interval: 0.992-3.672; P=0.053).

### Example 4. Confirmation of the Prognostic Use of VISTA in Idiopathic Pulmonary Fibrosis Using the Cox Proportional Hazard Model

To confirm the prognostic use of VISTA for idiopathic pulmonary fibrosis, multivariate analysis using the Cox proportional hazard model was conducted. The Cox proportional hazard model is a method used to analyze risk factors that affect survival rates. In this example, specifically, FVC (forced vital capacity), DLCO (Diffusing capacity of the Lung for CO), and antifibrotic agent administration were analyzed.

As a result (Table 4), the blood VISTA levels in patients with idiopathic pulmonary fibrosis (IPF) were found to independently predict the mortality of IPF patients, even after adjusting for age, FVC (Forced Vital Capacity), DLCO (Diffusing capacity of the Lung for CO), and antifibrotic drug administration (HR (hazard ratio) 1.001; 95% confidence interval [CI], 1.000-1.001; p=0.002).

TABLE 4

| | | | Multivariate Analysis | |
|---|---|---|---|---|
| | Univariate Analysis | | adgusted HR | |
| Classification | HR(95% CI) | p value | (95% CI) | p value |
| Age | 1.041 (1.004- | 0.031 | 1.022 (0.980- | 0.315 |
| | 1.080) | | 1.066) | |
| FVC(%) | 0.919 (0.896- | <0.001 | 0.960 (0.933- | 0.005 |
| | 0.943) | | 0.988) | |
| DLCO(%) | 0.937 (0.918- | <0.001 | 0.958 (0.934- | 0.001 |
| | 0.956) | | 0.983) | |
| Treatment | | | | |
| modality | Reference | p value | Reference | p value |
| Steroid | 0.933 (0.383- | 0.879 | 0.747 (0.273- | 0.569 |
| | 2.273) | | 2.040) | |
| Anti-fibrotic | 0.209 (0.101- | <0.001 | 0.184 (0.079- | <0.001 |
| agent | 0.433) | | 0.430) | |
| VISTA | 1.001 (1.000- | 0.025 | 1.001 (1.000- | 0.020 |
| (pg/mL) | 1.001) | | 1.001) | |

* Multivariate analysis was adjusted for age, FVC (%), DLco (%), and treatment modality.

The above description of the present disclosure is for illustrative purposes, and those skilled in the art to which the present disclosure pertains will understand that the invention can be easily modified into other specific forms without changing the technical spirit or essential characteristics of the invention. Therefore, the embodiments described above should be understood as illustrative in all respects and not limiting.

### INDUSTRIAL APPLICABILITY

The present disclosure relates to a novel biomarker VISTA for the diagnosis or prognosis prediction of idiopathic pulmonary fibrosis patients. By analyzing the VISTA level in blood separated from a subject, it not only enables easy diagnosis and prognosis prediction of idiopathic pulmonary fibrosis, but also demonstrates excellent diagnostic and prognostic effectiveness. Therefore, it can be effectively utilized as a non-invasive method for diagnosing or predicting the prognosis of idiopathic pulmonary fibrosis, and thus has industrial applicability.

1-11. (canceled)

12. A method for treating idiopathic pulmonary fibrosis, the method comprising:
determining a V-domain Ig suppressor of T cell activation (VISTA) level in plasma separated from a subject and comparing the detected VISTA level with a VISTA level determined in plasma separated from a control group; and
when the VISTA level in the subject's plasma is increased compared to the VISTA level in the control group, administering a therapeutically effective amount of a therapeutic agent to a subject diagnosed with idiopathic pulmonary fibrosis.

13. The method of claim 12, wherein the administering is performed when
a mean VISTA level in the plasma of the subject is at least 1.76 times the VISTA level in the control group, and
a p-value for the mean VISTA level is less than 0.001.

14. The method of claim 12, wherein the administering is performed when a median VISTA level in the plasma of the subject is at least 1.6 times the VISTA level in the control group.

15. The method of claim 12, wherein the administering is performed when a maximum value of the VISTA level range in the plasma of the subject is at least 2.85 times the VISTA level in the control group.

16. The method of claim 12, further comprising
predicting a poor prognosis of idiopathic pulmonary fibrosis based on the VISTA level in the subject's plasma compared to the control group.

17. The method of claim 12, wherein the control group comprises an individual who has been diagnosed with idiopathic pulmonary fibrosis at least once and has been determined to be cured.

18. The method of claim 16, wherein a poor prognosis for idiopathic pulmonary fibrosis is predicted when the subject's plasma VISTA level is at least 320 μg/ml.

19. The method of claim 16, wherein the poor prognosis includes a prediction of death of the subject within five years.
